(19) United States
(12) Patent Application Publication
(10) Pub. No.: US 2012/0150081 A1
(43) Pub. Date: Jun. 14, 2012

(54) SUPPORT UNIT FOR PHYSICALLY HANDICAPPED PATIENTS

(76) Inventor: Aslan Ali Pirli, Gebze (TR)

(21) Appl. No.: 13/203,136

(22) PCT Filed: Feb. 11, 2010
(86) PCT No.: PCT/TR2010/000032

§ 371 Date: Mar. 2, 2012

(30) Foreign Application Priority Data
Feb. 26, 2009 (TR) 2009/01453

(51) Int. Cl. A61F 5/44; A61G 7/02; A61H 1/00

(52) U.S. Cl. 601/49; 604/327; 5/604

(57) ABSTRACT

The invention is about the support unit for the physically handicapped patient that is used for the unconscious or conscious physically handicapped patient, fulfills the patient's needs automatically or through manually controlled directions, turns into a bed or wheeled handicapped chair when wished, ensures that the patient does physical movements, provides the blood circle by making massage under the lower base of the foot through the vibration of the step under the patient's feet, ensures that the patient defecates depending on the equipments under the seat section (22) of the chair and that defecation is transferred to the sewage system wholly automatically and hygienically without human touch, gaining the patient the freedom of movement.

The invention is about a support unit for physically handicapped patients which is used for conscious or unconscious physically handicapped patients, provides the patient's needs through automatic or manual control, is turned into a bed or a wheeled chair for physically handicapped patients, ensures that the patient performs physical acts, enables blood circulation by massaging to the feet's lower base through the vibration of the step under the patient's feet, ensures that the patient defecates and the dejection is transferred to the sewage system wholly automatically without human touch hygienically depending on the equipment under the seat section (22) of the chair and provides the patient with free scope.

Man kinds have not fulfilled their vital needs, like defecation, by themselves once or few times all their life because of the incapability of physical movement. It is very demanding to be helped and cleaned, because of the inadequate movement ability, by the family members or nursemaids. These kinds of people generally attached to one room cause their relatives to suffer pain and the people having this kind of patients have great troubles. That situation results in a decrease in those people's life quality. In the same way, the reality that the patients turn into trouble for their relatives is a source of sorrow for the patients themselves which causes psychological issues for them.

The support unit for physically handicapped patients saves the unconscious patients and the physically handicapped patients, who are the most serious cases in need of care, from being wholly depended on another person. The invention is ideal for using in fulfilling the needs of the elder, the bedridden, the handicapped and the paralyzed who are deprived of the movements that will fulfill their normal needs and who are in need of nursing.

### THE EXPLANATION OF THE INVENTION

The invention consists of those sections: the multi-functional underpants (17) which is fastened on the patient's body by dividing the gluteus and centering the anus without touching upon the periphery of anus, has electronic and electro mechanic equipment, is multi-layered and does the hygienic cleaning of the patient, the waste reserve storage (19) which, when fastened on the patient, removes the waste as soon as the waste leaves the patient's body, in an entirely controlled way without contaminating the patient's body and the surroundings, the waste transfer station (30) which can transfer the waste, to the sewerage system, automatically by moving with the help of sensors in the body of the unit as a result of fullness of the waste reserve storage (19).

### Multi-Functional Underpants:

It is designed for unconscious physically handicapped patients. It is an underpants which is attached to the unit through equipment existent in it in order to ensure that the patient fulfill his/her detection need and functions like hygienic cleaning without problem and does not limit the movements of the patient by the support of the unit equipments.

The inner surface level (1) of the part of the multi-functional underpants (17) that touches on the patient's body is of antibacterial quality and is covered with flexible material and cloth that does not affect blood circle. The multi-functional pant (17) is designed as to divide the gluteus and grab the patient's body ergonomically. It is worn and fixed to the patient by being arranged for every size through demounted additions.

The multi-functional pant (17) is consisted of two levels. In the middle of the first inner surface level (1) which touches on the patient body, there is a proper-sized hole (2) which is suitable for the middle of the anus and surrounds the anus. That hole, at the same time, centers waste removal pump (5) and, being attached to that, the strait (7) part which is a waste entry part to the anus, and supports them. There are cabins which are divided into levels (6) that constitute proper distance between the outer surface of the multi-functional pant's first inner level (1) that touches on the body and the inner surface (4) which is fixed to the second outer level (3). The aim of that distance is to constitute cabin for protecting the electromechanically equipped waste removal pump (5) from the pressure that the patient makes on the pant. The waste entry strait (7) of the mechanically equipped waste removal pump (5) is steered through the hole (2) which is in the middle of the multi-functional pant's (17) first inner level (1) that touches on the patient's body and which is suitable for the middle of the anus.

The waste entry strait (7) of the electro mechanic waste removal pump (5) ensures that the waste which comes out of the anus is transferred to the pump without contaminating the patient and his/her surroundings and through that way, the hygienic cleaning of the anus is performed. That waste entry strait (7), which surrounds the anus, is close to the anus but does not touch on the anus, and belongs to the waste removal pump (5), is of cylindrical shape in the width of the anus' opening diameter. The edges of the point (8), which belongs to the waste entry strait (7) of the waste removal pump (5) and touches on the anus' surroundings, are flexible and thin. That point is made from silicone or similar hygienic flexible material that does not hurt or irritate anus's surroundings during the touch on the edges of the anus (as shown in the FIGS. 1, 2, 3 and 5).

There are a micro camera fixation channel (9), a waste control sensor fixation channel (10), an air entry fixation channel (11), a water entry fixation channel (12), a light source, which enlightens the surroundings of the anus, fixation channel (13) on the outer edge of electromechanical waste removal pump's 5) waste entry strait (7) (FIG. 5). The equipments that will be mounted on those fixation channels are alerted by the sensor as soon as the wastes come out of the patient's body. Electromechanically equipped pump engine (31) operates after the latch (14), which closes the gap between, in the multi-functional pant (17), the waste entry strait (7) of the electro mechanic waste removal pump (5) and the hole (2) part surrounding the anus, is unbolted by the electro mechanism (15) that activates the latch. The latch (14), after the defecation finishes, by closing the waste entry strait (7) between anus and the pump, cuts the connection between the waste removal pump (5), the waste storage in the unit and anus. However, electromechanically equipped waste removal pump (5) has self-cleaning equipment through sprinkler water system. Thanks to the equipment, it is ensured that the surroundings of the anus remain hygienic by the out of the operation between the wastes that may be remained in the pump and the anus and also the cut of the smell transfer. Thus, the latch (14) is an apparatus that is designed with the aim of providing the anus with hygienic atmosphere. The latch (14) or figures of design is opened or closed through mechanisms like valves. When the wastes leave the body, the pneumatic diaphragm (16), which swells with the current air under the waste removal pump (5), through swell mechanic movement or electro mechanism, by raising the waste removal pump (5) up towards the anus and ensuring that the point (8) of the pump's upper waste entry strait (7) that touches on the surroundings of the anus touches on the surroundings of the anus (2) with proper pressure, hinders the leak of the wastes outside. Except those practices, if the patient is in rest position, the pneumatic diaphragm (16) that swells with the current air under the waste removal pump (5), by raising the waste removal pump up towards the anus with the swell mechanic movement or electro mechanism, by perpetuating the touch of the anus on its surroundings is for supporting the entry of the leaks like liquid that may come from anus into the waste removal pump (5). That precaution is designed in order to hinder the wastes from contaminating the body. When there is a change in the position of the patient, meaning the upright or sitting position, the pneumatic diaphragm (16) that swells with the current air under the waste removal pump (5), with the swell mechanic movement or electro mechanism, by replacing the waste removal pump (5), ensures that the connection of the pump's point (8) that touches on the surroundings of the anus with the body is cut.

Whatever position the patient is in, rest, upright, sitting, lying on back or lying on both sides, the multi-functional pant (17) with the help of its technical equipment and unit, fulfills the patient's defecation need automatically and does not limit the movements of the patient. The multi-functional pant (17) transfers the wastes to the waste reserve storage (19) through a pipe (18). At the same time, because the electromechanically equipped waste removal pump (5) is also on, solid wastes are transferred to the waste reserve storage (19) which is available in the unit.

### Mobile Multi-Functional Pant:

The mobile multi-functional pant (17) is designed for astronauts, little children and physically handicapped traveling patients etc. It is a mobile multi-functional pant (17) which is designed for fulfilling the needs of those people who travel for various reasons and which has an electronic equipment to practice the necessary instructions that are for the electro mechanic mechanisms on it to fulfill the procedures. It performs an entirely hygienic cleaning and it transfers the wastes, without contaminating them on the person's body, to the current storage in it. The mobile multi-functional pant (17) does not limit the movements of the people wearing it.

The inner surface level (1) of mobile multi-functional pant (17)'s part that touches on the body of the patient is covered with flexible material and cloth which do not make the patient sweat, affect the blood circle and are of antibacterial quality. The multi-functional pant (17) is designed as to divide the gluteus and grab the patient's body ergonomically. It is worn and fixed to the patient by being arranged for every size through demounted additions.

The mobile multi-functional pant (17) is consisted of two levels. In the middle of the first inner level (1) which touches on the patient body, there is a proper-sized hole which is suitable for the middle of the anus and which surrounds the anus. That hole, at the same time, centers waste removal pump (5) and, being attached to that, the strait (7) part which is a waste entry part to the anus and supports them. There are cabins which are divided into levels (6) that constitute proper distance between the outer surface of the mobile multi-functional pant's inner surface level (1) that touches on the body and the inner surface (4) which is fixed to the second outer level (3). The aim of that distance is to protect the electromechanically equipped waste removal pump (5) from the pressure that the patient makes on the pant and to constitute cabins for waste reserve storage (49), clean water storage (50), DC energy battery (51). The waste entry strait (7) of the mechanically equipped waste removal pump (5) is steered through the hole which is in the middle of the mobile multi-functional pant's (17) first inner surface level (1) that touches on the patient's body and which is suitable for the middle of the anus. The waste entry strait (7) of the waste removal pump (5) ensures that the waste which comes out of the anus is transferred to the pump without contaminating the patient and his/her surroundings and through that way, the hygienic cleaning of the anus is performed. That waste entry strait (7), which surrounds the anus, is close to the arms but does not touch on the anus, of the waste removal pump (5) is of cylindrical shape in the width of the anus' opening diameter. The edges of the point (8), which belongs to the waste entry strait (7) of the waste removal pump (5) and touches on the anus' surroundings are flexible and thin. That point is made from silicone or similar hygienic flexible material that does not hurt or irritate anus's surroundings during the touch on the edges of the anus (as shown in the FIGS. 1, 2, 3 and 5).

There are a micro camera fixation channel (9), a waste control sensor fixation channel (10), an air entry fixation channel (11), a water entry fixation channel (12), a light source, which enlightens the surroundings of the anus, fixation channel (13) on the outer edge of electromechanical waste removal pump's waste entry strait (7) (FIG. 5). The current electronic control center (48) in the mobile multi-functional pant (17) is alerted by the sensor as soon as the waste comes out of the patient's body. The equipments that will be mounted on those fixation channels and the consistent electronic control center (48) in the mobile multi-functional pant (17) are alerted by the sensor as soon as the wastes come out of the patient's body. Upon the alert, the electronic control center (48) operates. After the latch (14) which closes the gap between, in the mobile multi-functional pant (17), the waste entry strait (7) of the electro mechanic waste removal pump (5) and the hole (2) part surrounding the anus, is unbolted by the electro mechanism (15) that activates the latch, the electronically equipped pump engine (31) operates, the pneumatic diaphragm (16) through swell mechanic movement by raising the waste removal pump (5) up towards anus and by ensuring that the point (8) of the upper waste entry strait (7) which touches on the surroundings of the anus (2) touches on the surroundings of the anus with a proper pressure hinders the leak of waste outside. However, electromechanically equipped waste removal pump (5) has self-cleaning equipment through sprinkler water system. It is ensured that the surroundings of the anus remain hygienic by the cut of the operation between the wastes that may be remained in the pump and the wastes that is in the waste reserve storage (49) of the multi-functional pant (17) and the anus and also the cut of the smell transfer. Thus, the latch (14) is an apparatus that is designed with the aim of providing the anus with hygienic atmosphere. The latch (14) or other figures of design is opened or closed through mechanisms like valves.

The electronically equipped waste removal pump (5) transfers the solid wastes to the current waste storage (49) of the mobile multi-functional pant (17). Then it performs the hygienic cleaning of the patient. The waste transfer mechanism (29) that is settled under the waste storage (49) of the mobile multi-functional pant (17) has technical qualities that are similar to the multi-functional pant's but here the waste reserve storage is the waste reserve storage of the mobile multi-functional pant. When the wastes are full of in the waste reserve storage (49) of the mobile multi-functional pant (17), the waste reserve storage gives caution warnings through the current level sensor in it.

It is ensured that the mobile multi-functional pant, like in the multi-functional pant, is attached to the unit in order to empty the wastes in the waste reserve storage (49) of the mobile multi-functional pant (17) and transfer the wastes to the waste transfer mechanism (29).

The mobile multi-functional pant (17) with the support of its current technical equipment automatically provides the needs of the patient. It transfers the wastes to the waste reserve storage (49), during those procedures, it does not contaminate the defecation wastes of the people anywhere but the opening of anus (2) and it does not limit the movements.

### Waste Transfer Mechanism:

Waste transfer mechanism (29) is settled in the unit and transfers the wastes in the waste reserve storage (19), where the wastes that are removed from the patient are collected, to the sewage system, through its mechanical equipment, by the directions of the unit, by attaching waste transfer station (30) automatically to the sewage system.

The waste transfer mechanism (29) takes place under the patient in the cabin of the unit (23). Waste transfer mechanism (29) has alignment channels (47) that enable the waste transfer mechanism to clamp to the waste transfer station (30). Those channels ensure that the waste transfer mechanism (29) is clamped and separated with the waste transfer station (30) by activating it through engine with a reducer (53) or pneumatic mechanic movements. Waste transfer mechanism (29) has a pipe (18) through which the multi-functional pant (17) attached on the patient removes the waste from the patient and has waste reserve storage (19) where the wastes are collected. The wastes are stored in the waste reserve storage (19). The gases of wastes that are stuck because of the wastes in the waste reserve storage (19) are filtered with gas filter (32) and through that way the smell is handled. There is a waste transfer pump (33), which is fixed to the storage, which mixes the wastes and transfers to the station, under the waste reserve storage (19). The waste reserve storage (19) is funnel-shaped and curved and has a very slippery surface in order to ensure that the wastes flow to the waste entry opening part of the waste transfer pump (33). Those qualities of the waste reserve storage (19) hinder the accumulation of the wastes on the edges and ensure that the waste transfer pump (33) flows to the waste entry opening. It alerts the unit through waste level sensor (34) when the waste reserve storage (19) is full of wastes. The unit notifies the warning of the waste reserve storage (19) through voice or light to the people outside the unit and it gives the warning for a direction to the waste transfer station (30). The waste transfer pump (33) in the waste transfer mechanism (29), until the directed unit automatically takes it to the waste transfer station (30) and stops in the line of clamping, by continuing operating, circulating the wastes in the inner waste circulation channel (35), transforming through the waste reserve storage (19), diluting the viscosity of the wastes that are subsided, providing the wastes with fluidity, simplifies the pumping of the wastes to the waste transfer station (30). The mechanism that operates the clamp that is fixed by passing before the waste transfer pipe (37) through which the waste transfer pump (33) pumps the wastes is a mechanism (36) which has one entry and two exits and through which the wastes, divided into two, pass. One (37) of the waste transfer pipes of the mechanism (36) operating the valve, which are divided into two, transfers the wastes to the station, the other (37) circulates the wastes by returning back to the inside of the storage. There is a waste redirecting clamp (38) in the point of the part where the pipes meet, which is controlled by the unit, has mechanical movements and will close the one of holes on the pipe on its both sides. The waste redirecting clamp (38) ensures that the wastes, which the waste transfer pump (33) transfers, circulates by the direction it gets from the unit by opening the entry of one of pipes and closing the entry of the other or ensures that the clamping occurs for its pumping to waste transfer station (30). If the clamping of the waste transfer mechanism (29), which is attached to the unit, to the waste transfer station (30) becomes unsuccessful, the procedure does not continue. If the clamping becomes successful, the unit gives direction to the waste transfer mechanism (29) to make the wastes transfer to the waste transfer station (30).

Waste transfer mechanism (29), by operating upon the direction it gets, from the waste transfer station (30) to the unit, with clean water pipe (39), transfers clean water, transfers energy to the station through DC energy transfer battery (40) and it performs automatically that the wastes are transferred to the waste transfer station (30). The waste transfer pump (33) current in waste transfer mechanism (29) with the help of sprinklers current in it, after the procedure of waste transfer, cleans itself through washing. After all those procedures finish, waste transfer mechanism (29) alerts the unit with the warning of the procedures' ending. All the procedures that waste transfer mechanism (29) performs are performed without human intervention automatically.

### Station of Waste Transfer to the Sewage System:

As shown in the FIGS. 16 and 17, waste transfer station is settled under the closet. Waste transfer station (30) is designed for settling connection with the sewage system, clamping to the unit, and without any problems transferring the wastes transferred from the unit to the sewage system. Waste transfer station (30) has equipments that transfer clean water to the unit and clean themselves.

Waste transfer station (30) is an equipment that is out of the unit and combines the unit. There are two current solenoids in the waste transfer station (30). One of those solenoids is attached to the main water system through a clean water pipe (39). The other one is that transfers water to the sprinkler (41) which washes the parts of the waste transfer station through which wastes transfer to the sewage system. Those solenoids work through the energy transfer with DC energy battery (40) by the unit during the clamping of the unit with waste transfer station (30) and through the direction the unit gives. Waste transfer station (30), by the dismembering of the closet fixation screws (43) that fix the closet (42) that has a connection with the sewage system in the places where the patient takes place like toilet, bathroom etc, replaces the closet (42) and the waste pipe part of the station (44) replaces the sewage system pipe (45). Then the closet (42) is settled on the waste transfer station (30). The closet (42), with the screws (43) of enough length that are given with waste transfer station (30), is fixed on the ground with the waste transfer station. In addition to that, if there is not closet (42), the waste transfer station (30) has equipments that will be mounted on the places that are near to the sewage system etc. or that are attached to the sewage system. In order for the waste transfer station (30) to help the unit clamp by performing transferring the wastes that are transferred from the unit, there is an alignment panel (52) which is attached with the waste transfer station (30), is mounted on the ground and has two channels. The alignment channels (47) of the unit alignment panel (52) aligns the unit to the waste transfer station (30) in order to ensure that the two front wheels of the unit pass and make the unit clamp to the waste transfer station (30) without any problems. While, after the clamping, the alignment channels complete the transfer of the wastes, that have been transferred to the waste transfer station (30) from the unit, to the sewage system without contaminating the surroundings, the waste transfer station (30), upon the direction by the unit, by opening the clean water pipe (39) solenoid that is one of the solenoids consistent in the waste transfer station, transfers the clean water to the storage of the unit. After the procedure of the waste transfer pipe (37) is finished, by opening the sprinkler (41) solenoid finished the cleaning of the target parts of the station. Thus, the unit, by performing all these procedures automatically, leaves the unit.

### Squatting Position:

The support unit for conscious or unconscious physically handicapped patients characterized in that; it brings the patient to the squatting position while it fulfills the patient's defecation need and supports that the wastes are easily come out of the patient's body and the patient is relieved.

While the patient is fulfilling the defecation need, whatever position the patient is, as soon as the wastes leave the body, with the warning of the waste control sensor (10) current in the multi-functional pant (17), the unit brings the patient to the squatting (chair) position. Then the step (20) under the patient's feet and mobile step frame (28), to which the step is attached to, through the help of the mechanism by rising up brings the patient to the squatting position. That mechanic movement leads the patient's knees to make pressure on the abdominal region of the patient so it performs the cleaning of the intestines by helping the wastes transfer out of the body of the patient.

### Bed Position:

The support unit for conscious or unconscious physically handicapped patients characterized in that; it turns into a bed of standard bed size when wished and turns into a chair when wished. By these means, it becomes possible that the patient is wandered in various places with the unit.

When the patient is in bed position, the lateral wings (21) are of collapsible quality. The angles of the lateral wings (21), in order to hinder the patient from falling off the bed, are designed as to arrange in desired levels according to the consciousness level of the patient. Thus those lateral wings ensure that the unit can turn into a large bed after an opening and can get small enough to get through the doors. The equipments, on which there are manually controlled equipments and on which the patient put his/her arms when the patient uses the unit as a chair, are demounted on the lateral wings (21). When the unit turns into a bed, the pillow (24) support at the head side is curved in an angle that will provide the pillow need of the patient. When a direction is given in order to turn the unit into chair position, the unit by curving its lateral wings (21) inside again, turns into a mobile chair which has enough sizes to get through the doors. That situation gains the patient the freedom of sleeping comfortably in standard bed sizes in any place. The lateral wings (21), by opening in wished angles with mechanic movement, provide the quality of conversion to bed in standard bed sizes.

### Provision of Physical Movement:

The support unit for conscious or unconscious physically handicapped patients which is designed for making the patient do physical movements characterized in that; it aims to hinder the constitution of bedsores on the body of the patient by fastening the blood circle of the patient through erecting the patient in different slants.

It ensures that the legs of the patient are curved from the knees and thus the legs move by keeping the patient in different slants and in upright position (25), by mobilizing the step (20) under the patients' feet up and down. There are safety belts (26) settled on the knee and chest which limits the unwelcome movements and hinders the patient from falling from the bed while the physical movements are done. The safety belts (26) support the patient to do movements in upright or other slants. The safety belt (26) that is attached to the knees of the patient is related to the step (20) on which the legs of the patient steps. The safety belt (26) and the step (20) do the movements together. The safety belt (26), designed differently for the knees, ensures that the knees of the patient are curved by moving with the step (20) simultaneously when the step moves upwards and by relaxing simultaneously ensures that the knees of the patient turn back to its former straight position when the step (20) moves downwards. The amount of pressure, which is made to the parts of the bed on which the patient touches, is proportionate with the slant of the body of the patient in vertical position (27). Thus the amount of pressure lessens when the patient gets closer to the upright position (25) from vertical position (27) and thanks to the movement the blood circle of the body gets normal. Thus the negative results like bed sores which are resulted from the pressure on the body are hindered through supporting the blood circle of the patient.

### Massaging Under the Foot:

The support for the physically handicapped which is designed for unconscious physically handicapped patients characterized in that; it ensures that it fastens the blood circle of the patient by massaging under his/her feet in various angles and it relaxes the muscles of the patient. The step, on which the patient steps his/her feet by creating vibration, through the electro mechanic equipment it has, massages under the feet of the patient and ensures that the patient relaxes.

### BRIEF EXPLANATIONS OF THE FIGURES

FIG. 1: The back view of the inner surface of the multi-functional pant (17) in which the inner surface is fixed on the body of the patient and the surroundings of the anus.

FIG. 2: Isometric sectional view of the multi-functional pant.

FIG. 3: Upper isometric sectional view of the multi-functional pant (17) fixed on the patient.

FIG. 4: Sidelong general sectional view of the multi-functional pant (17) fixed on the patient.

FIG. 5: Sidelong detailed sectional view of the waste removal electro mechanic pump (5).

FIG. 6: Isometric sectional view of Latch (14).

FIG. 7: Upper view of Latch (14).

FIG. 8: Isometric general sectional view of the waste reserve storage.

FIG. 9: Front view of the waste reserve storage.

FIG. 10: Sidelong view of the unit in bed position.

FIG. 11: The front view of the collapsible lateral wings of the unit.

FIG. 12: The sidelong view of the up and down movement of the step where the unit is in the chair position.

FIG. 13: The sidelong view of the different angled positions of the patient fixed on the bed.

FIG. 14: The sidelong view of the physical movement position of the patient in vertical position.

FIG. 15: The upper sectional view of the bypass contrivance of the waste reserve storage.

FIG. 16: The sidelong isometric view of the waste transfer station.

FIG. 17: The front isometric view of the waste transfer station.

FIG. 18: The sidelong isometric view fixed on the unit profile of the waste transfer station fixed on the closet.

FIG. 19: The front isometric view of the unit alignment panel.

FIG. 20: The upper view of the unit alignment panel.

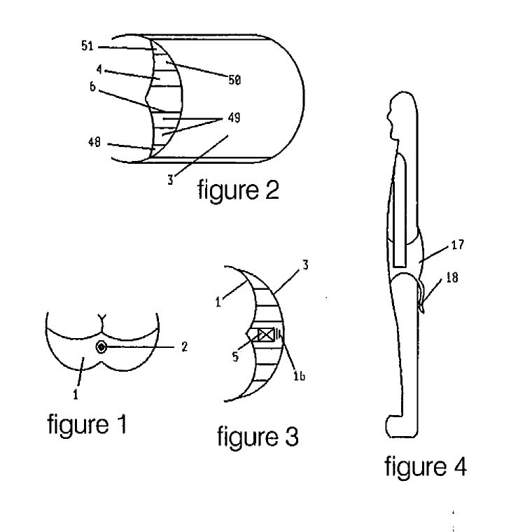

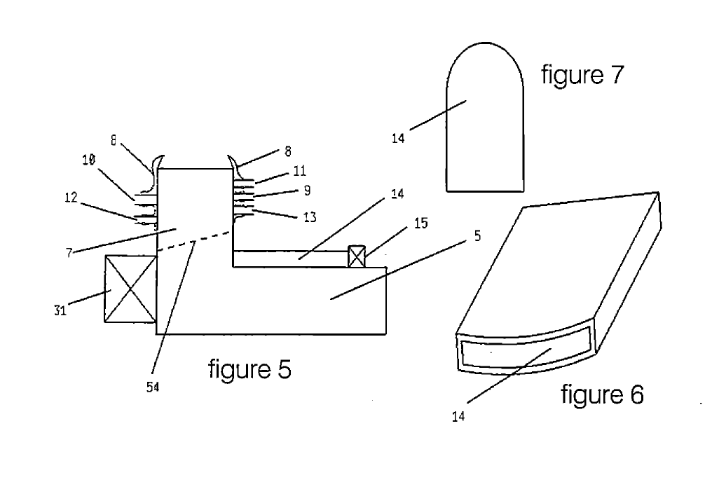

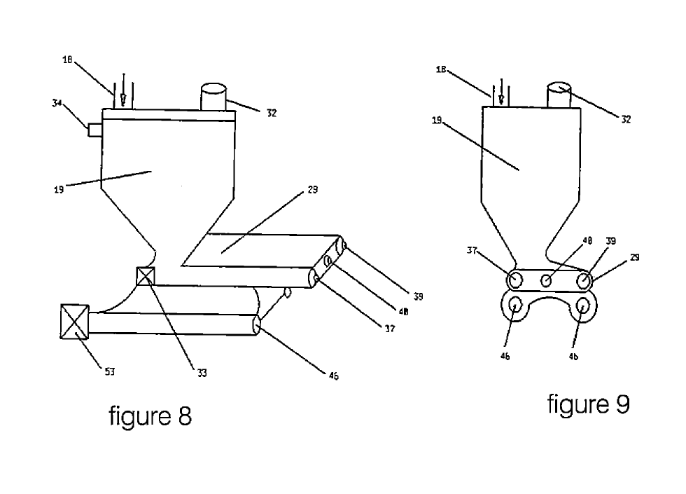

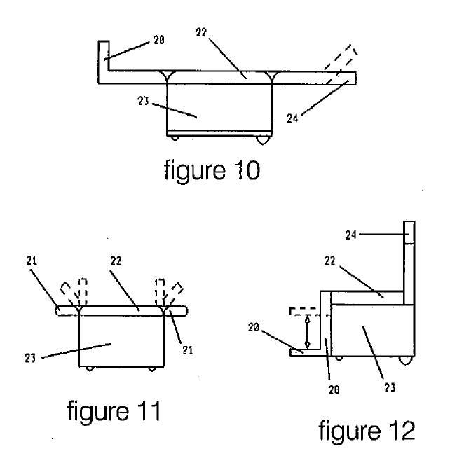

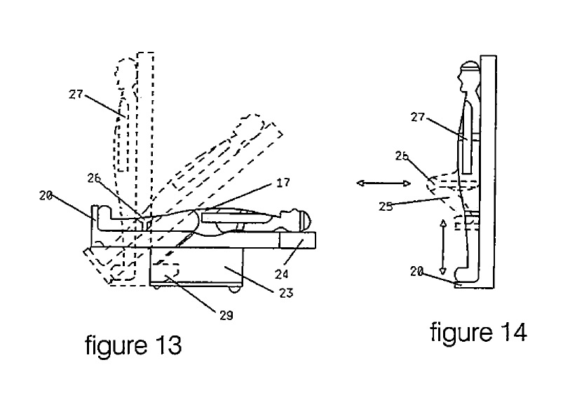

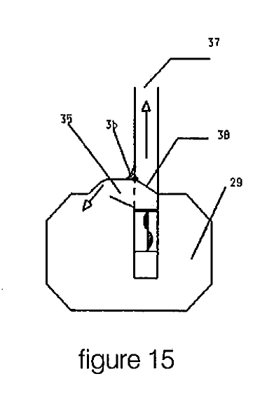

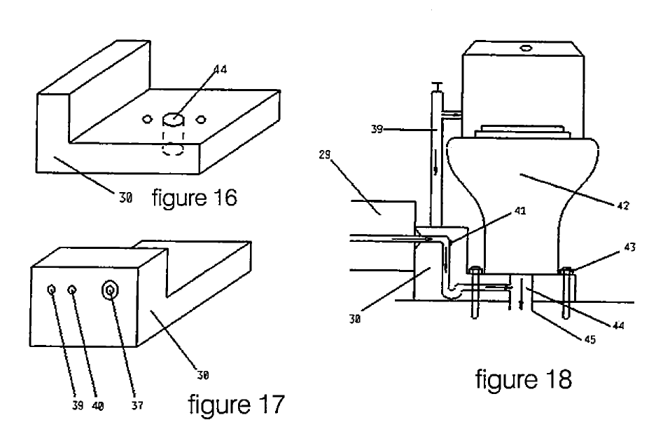

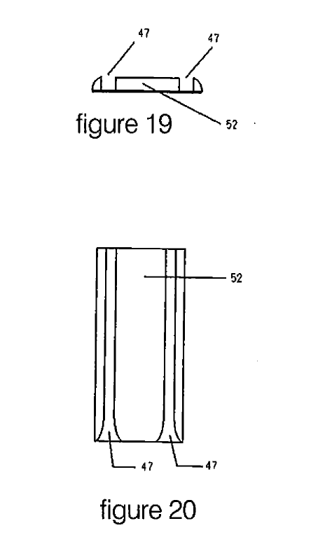

### The Explanations of the Parts that are Consistent in the Unit for the Physically Handicapped Patients

1. The inner surface level that touches on the patient's body by dividing the gluteus
2. The hole surrounding the anus of the patient
3. The level having multi-divisional cabins
4. Clean water division that will be used for the mobile multi-functional pant
5. Waste removal electro mechanic pump
6. The levels constituting distances of enough length
7. The strait which is the waste entry part of the waste removal pump (5)
8. The point of the waste removal pump (5) that touches on the surroundings of the anus
9. Micro camera fixation channel
10. Waste control sensor fixation channel
11. Air entry fixation channel
12. Water entry fixation channel
13. Light source fixation channel
14. The latch
15. Electro mechanism operating the latch
16. Pneumatic diaphragm
17. Multi-functional pant
18. The pipe transferring the wastes of the multi functional pant
19. Waste reserve storage
20. The step of the unit moving up and down
21. The collapsible lateral wings of the unit
22. The seat section of the unit
23. The cabin of the unit
24. The pillow support of the unit
25. Upright position
26. The mobile knee-guard belt of the patient
27. Vertical position
28. Mobile step chassis
29. The waste transfer mechanism of the unit
30. The waste transfer station
31. The waste transfer electro mechanically equipped pump engine
32. Gas filter
33. The waste transfer pump
34. The waste level sensor
35. The waste inner circulation channel
36. The mechanism operating the clamp
37. The waste transfer pipe
38. The waste direction clamp
39. Clean water pipe
40. DC energy transfer battery
41. The sprinkler washing the parts of the unit through which the wastes go to the sewage system
42. The closet
43. The closet fixation screws
44. The station waste pipe part
45. The sewage system pipe
46. The shaft providing the back and forth movement of the waste reserve storage
47. The alignment of the unit to the waste transfer station channels
48. The current electronic control centre in the mobile-multifunctional pant
49. The waste reserve storage of the mobile multi-functional pant
50. The clean water storage of the multi-functional storage
51. DC battery cabin of the multi-functional pant
52. The unit alignment panel
53. Waste transfer mechanism electro mechanic equipped engine with reducer
54. Pump (5) strait clasp

1. The support unit for the physically handicapped patient characterized in that;
a) Multi-functional pant (17) which consists of a hole (2) in the middle of the first inner surface level (1) that touches on the patient's body, aligning with the middle of the anus and surrounding it, centering electro mechanic waste removal pump (5) and depended on that, the strait (7) part that is the waste entry part to the anus, cabins divided into levels (6) constituting long enough distances between the outer surface of the first inner surface level (1) and the inner surface (4) of the second outer surface level (3), a micro camera fixation channel (9), a waste control sensor fixation channel (10), an air entry fixation channel (11), a water entry fixation channel (12), a light, that enlightens the surroundings of the anus, fixation channel (13) on the outer edge of the waste entry strait (7) of electro mechanic waste removal pump (5).
b) The waste transfer mechanism (29) which has a pipe (18), settled in the cabin (23) of the unit under the patient, removing the wastes from the patient, a waste reserve storage (19) where the wastes are collected, under that storage waste transfer pump (33) fixed on the storage mixing the wastes and transferring to the station, alignment channels (47) performing the clamping and separating situation with the waste transfer station, gas filter (32) for stocked waste gases, waste level sensor (34), waste transfer pipe (37) where the wastes are pumped, mechanism (36) operating the valve which is fixed through by passing front of it, waste direction valve (38) which is on the tip of the part in the place where the waste transfer pipe (37) divided into two is combined, controlled by the unit, which has mechanical movements, will close one of the holes of the pipes on its both sides.
c) The waste transfer station (30) which, by providing the connection of the unit with the sewage system, transfers the wastes, that are transferred from the unit, to the sewage system, has two solenoids one of which transfers water to the clean water pipe (39) attached to the city water, and one of which transfers water to the sprinkler (41) that washes the parts of the station where the wastes go to the sewage system, which has a station waste pipe part (44) that is fixed, instead of the closet (42), on the sewage system pipe (45) on the ground.

2. The multi-functional pant mentioned in the claim 1 characterized in that; the distance that is constituted between the outer surface of the first inner level (1) touching on the body and the inner surface (4) of the second outer level (3) is for creating cabins in order to protect the electro mechanically equipped waste removal pump (5) from the pressure that the patient makes on the pant.

3. The multi-functional pant mentioned in the claim 1 characterized in that; the waste entry strait (7) of the electromechanically equipped waste removal pump (5) gets closed to the anus by being steered through the hole (2) that is in the middle of the first inner level (1), that touches on the body of the patient, and that aligns with the middle of the anus.

4. The multi-functional pant mentioned in the claim 1 characterized in that; it includes a waste removal pump (5) removing the wastes that comes out of the anus through waste entry strait (7) without touch on the body of the patient.

5. The multi-functional pant mentioned in the claim 1 characterized in that; the waste removal pump (5) includes a waste entry strait (7) which surrounds the anus, is close to anus but does not touch to the anus, is of cylindrical shape that has a length of the opening diameter of the anus.

6. The multi-functional pant mentioned in the claim 1 characterized in that; it includes a latch (14) that closes the space between the waste entry strait (7) of the electro mechanic waste removal pump (5) and the hole (2) surrounding the anus, that cuts the connection between the waste removal pump (5), the storage in the unit and the anus by closing the waste entry strait (7) between the anus and the pump after the removal process finishes, that moves through clasp mechanism or electro mechanism (15).

7. The multi-functional pant mentioned in the claim 1 characterized in that; it includes a pneumatic diaphragm (16) that swells with the air consistent under the waste removal pump (5) after the wastes leave the body, that hinders the leak of wastes outside by ensuring that the point (8) of the upper waste entry strait (7) part of the pump that touches on the anus touches on the surroundings of the anus (2) with proper pressure through raising the waste removal pump (5) up.

8. The multi-functional pant mentioned in the claim 1 characterized in that; the inner surface (1) of the first inner level that touches on the patient's body is covered with flexible material and cloth that does not affect blood circulation.

9. The multi-functional pant mentioned in the claim 1 characterized in that; the pneumatic diaphragm (16), the waste removal pump (5), the point (8) of entry strait, when the patient is in bed position, by touching on the surroundings of the anus automatically, hinder the leak of the involuntary liquids etc. that may happen in the anus to the unwanted regions.

10. The waste transfer mechanism mentioned in the claim 1 characterized in that; it has electromechanically equipped engine (53) with reducer or alignment channels (47) that are operated through pneumatic mechanic movements and ensures that the mechanism clamps to or separates from the waste transfer station (30).

11. The waste transfer mechanism mentioned in the claim 1 characterized in that; the waste gases that are stucked in the waste reserve storage (19) because of the wastes are filtered through a gas filter (32).

12. The waste transfer mechanism mentioned in the claim 1 characterized in that; it includes the waste transfer pump (33) that is fixed onto the storage under the waste reserve storage (19), mixes the wastes and transfers to the station.

13. The waste transfer mechanism mentioned in the claim 1 characterized in that; the waste reserve storage (19) is of slanted infundibular shape and has a slippy surface in order to ensure that the wastes flow into the waste entry opening part of the waste transfer pump (33).

14. The waste transfer mechanism mentioned in the claim 1 characterized in that; it alerts the unit through the waste level sensor (34) when the wastes fill the waste reserve storage (19).

15. The waste transfer mechanism mentioned in the claim 1 characterized in that; the waste transfer pump (33), until the directed unit stops at the clamping level by taking itself automatically to the waste transfer station (30), simplifies for itself the pumping, by continuing working and providing the wastes with liquidity, through diluting the viscosity of the subsided wastes, by circulating the wastes in the inner waste circulation channel (35) and transforming it in the waste reserve storage (19).

16. The waste transfer mechanism mentioned in the claim 1 characterized in that; the waste transfer pipe (37), which is one part of the mechanism (36) that operates the clamp and is divided into two, transfers the wastes into the station while the other part, the waste inner circulation channel (35) circulates the wastes by returning into the storage.

17. The waste transfer mechanism mentioned in the claim 1 characterized in that; it includes a waste direction clasp (38) which is on the point of the part where the waste transfer pipe (37) and the waste inner circulation channel (35) is combined, controlled by the unit, has mechanical movements, circulates the wastes by opening one hole and closing the other or ensures that the clamping occurs in order to pump to the waste transfer station (30).

18. The waste transfer mechanism mentioned in the claim 1 characterized in that; it ensures that it transfers water to the clean water storage in the unit with the clean water pipe (39), after the clamping of the waste transfer mechanism (29) to the waste transfer station (30), through transferring DC energy, from the unit to the station, with the DC energy battery (40) and that it transfers the wastes to the waste transfer station (30).

19. The waste transfer mechanism mentioned in the claim 1 characterized in that; the waste transfer pump (33), through the help of the sprinklers (41), current in itself and the station, after the waste transfer procedure, clean the mechanism and the waste channels by washing.

20. The waste transfer station mentioned in the claim 1 characterized in that; the solenoids work through the transferred energy by the unit with the DC energy transfer battery (40) and through the instruction by the unit during the clamping of the unit with the waste transfer station (30).

21. The waste transfer station mentioned in the claim 1 characterized in that; the waste pipe part of the station (44) is replaced instead of the closet (42) by the closet fixation screws' (43), that fixes the closet (42) that, where it takes place, has a connection with the sewage system, being dismantled.

22. The waste transfer station mentioned in the claim 1 characterized in that; it includes a unit alignment panel (52) that helps the waste transfer station clamp to the unit in order to make the wastes that come from the unit transfer to the sewage system, fixed on the ground, has two channels, connected with the waste transfer station (30).

23. The waste transfer station mentioned in the claim 1 characterized in that; the alignment channels (47) of the unit alignment panel (52) which aligns the unit to the waste transfer station ensures that the two front wheels of the unit passes and the unit is aligned in order to make the unit clamp to the waste transfer station (30) without any problems.

24. The support unit for the physically handicapped patients mentioned in the claim 1 characterized in that; it brings the patient to the chair position through the warning of the waste control sensor (10) consistent in the multi-functional pant (17) as soon as the wastes leave the body while the patient is defecating in whatever position he/she is, then it brings the patient to the squatting position through raising up of the moving step and its moving chassis (28), to which the step (20) under the patient's feet is attached, with the help of the mechanism.

25. The support unit for the physically handicapped patients mentioned in the claim 1 characterized in that; it, when wished, turns into a bed of one-person standard bed size, when wished, turns into a chair, is of collapsible quality when the patient is in bed position in the unit, has arrangeable sizes in wished sizes, the lateral wings (21) on which the patient puts his/her arms and which is demounted on the manual control equipment, a pillow support (24) on its head part which can curve in an angle that will fulfill the needs of the patient.

26. The support unit for the physically handicapped patients mentioned in the claim 1 characterized in that; it ensures that the patient's legs are curved from the knees and thus the patient moves through keeping the patient attached to the unit in the different slants, in the upright position (25), and by moving up and down the step (20) under the patient's feet through mechanical movements.

27. The support unit for the physically handicapped patients mentioned in the claim 1 characterized in that; there are safety belts (26), placed at the knee and chest region, that hinders the patient from falling down the bed side, the safety belt (26) attached to the knees is related with the step (20) under the patient's feet, and the safety belt (26), attached on the knees, by moving proportionally with the step (20) when the step (20) moves up and down and thus by curving the patient's knees and turning the patient's knees into their former straight position by removing back when the step (20) moves down ensures that the patient does physical movements.

28. The support unit for the physically handicapped patients mentioned in the claim 1 characterized in that; the step (20), on which the patient steps, makes massage under the lower base of the feet of the patients who have lost the ability of physical movement, by the vibration, thanks to the electromechanical equipment it has.

29. The mobile multi-functional pant (17) characterized in that; it has a hole (2) that is in the middle of the first inner level (1) that touches on the patient, aligns with and surrounds the middle of the anus and centers the electro mechanical waste removal pump (5) and depended on that the waste entry part, the strait (7), to the anus, it has cabins that constitute certain distance levels (6) between the outer surface of the first inner level (1) and the inner level (4) of the second outer level (3), a micro fixation channel, a waste control sensor fixation channel (10), an air entry fixation channel (11), a water entry fixation channel (12), light source, that enlightens the surroundings of the anus, fixation channel (13) on the outer edge of the waste entry strait (7) of the electro mechanic waste removal pump (5).

30. The mobile multi-functional pant mentioned in the claim 29 characterized in that; The aim of the distance that is constituted between the outer surface of the first inner level (1) and the inner surface of the second outer level (3) is to create cabin in order to save the electromechanically equipped waste removal pump (5) from the pressure that is made on the pant by the patient and is to create cabin for the waste reserve storage (49), clean water entry (50), DC energy battery (51).

31. The mobile multi-functional pant mentioned in the claim 29 characterized in that; the waste entry strait (7) of the electromechanically equipped waste removal pump (5) is steered through the hole (2), that is in the middle of the first inner level (1) that touches on the body of the patient, and thus is closed to the anus.

32. The mobile multi-functional pant mentioned in the claim 29 characterized in that; it includes the waste removal pump (5) that removes the waste coming from the anus and the waste entry strait (7) without contaminating the patient and the surroundings.

33. The mobile multi-functional pant mentioned in the claim 29 characterized in that; it includes a waste entry strait (7) that is the point (8) of the waste removal pump (5), surrounds the anus, is close to the anus, but does not touch to the anus, is in the diameter of the anus's opening, cylindrical, flexible and thin.

34. The mobile multi-functional pant mentioned in the claim 29 characterized in that; It includes a latch (14) that closes the distance between the waste entry strait (7) of the electro mechanic waste removal pump (5) and the hole (2) surrounding the anus and that, by closing the waste entry strait (7) between the anus and the pump after the procedure finishes, cuts the connection between the waste removal pump (5) and the waste storage (49), and which removes through the clamp mechanism.

35. The mobile multi-functional pant mentioned in the claim 29 characterized in that; it includes a pneumatic diaphragm (16) that swells with the air consistent under the waste removal pump (5) when the wastes leave the body, through the swell mechanic movement or electro mechanism by raising the waste removal pump (5) up towards the anus ensures that the point (8) of the upper waste entry strait (7) of the pump touches on the surroundings of the anus (2) with the proper pressure and thus hinders the leak of the wastes outside.

36. The mobile multi-functional pant mentioned in the claim 29 characterized in that; the first inner level (1) of the part that touches on the patient of the body is covered with flexible material and cloth that is non-sweating and anti-bacterial.

37. The mobile multi-functional pant mentioned in the claim 29 characterized in that; electromechanically equipped waste removal pump (5), by removing the pump to the waste storage (49), gives a warning alert through the level sensor consistent in it when the wastes fill the storage.

38. The mobile multi-functional pant mentioned in the claim 29 characterized in that; it ensures, in order to remove the wastes in the waste reserve storage (49), that the waste reserve storage is attached to the unit like in the multi-functional pant so the wastes go through the waste removal mechanism (29).
